(19) United States
(12) Patent Application Publication
(10) Pub. No.: US 2010/0034880 A1
(43) Pub. Date: Feb. 11, 2010

(54) PHARMACEUTICAL COMPOSITIONS BASED ON A MICROEMULSION

(75) Inventors: Amnon Sintov, Omer (IL); Haim Levy, Raanana (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NANODERMA LTD, Raanana (IL)

(21) Appl. No.: 12/525,811

(22) PCT Filed: Feb. 5, 2008
(86) PCT No.: PCT/IL08/00155

§ 371 Date: Oct. 13, 2009

(30) Foreign Application Priority Data
Feb. 8, 2007 (IL) 181217

(51) Int. Cl. A61K 9/113; A61K 38/28

(52) U.S. Cl. 424/484; 514/3

(57) ABSTRACT

The invention provides a transdermal, transmucosal pharmaceutical composition suitable for substantially extra-vascular application of at least one biologically active substance to biological membranes of a mammal, comprising a pharmaceutical or cosmetic composition comprising propylene carbonate at least one oil or source of fatty acid or surfactant; and water; in combination with the at least one biologically active substance wherein the propylene carbonate is adapted to enhance the bioavailability of the at least one biologically active substance.

### FIELD OF THE INVENTION

This invention relates to pharmaceutical and cosmetic compositions for external administration and methods for their preparation.

### BACKGROUND OF THE INVENTION

Many known drugs and medications are currently delivered to human patients via injection or by oral administration. Injections cause discomfort to the patient. Oral administration is sometimes not efficient while resulting in intra- as well as inter-subject variations, and often leads to at least a partial loss of some of the drug due to enzyme activity in the gastrointestinal route.

There has thus been a drive to find new routes for drug administrations and compositions which allow efficient passage and high bioavailability of the drug.

One form of composition is delivery in a micro-emulsion. The term “microemulsion” was defined by Danielson and Lindman (Colloids Surfaces, 3:391, 1981) as follows: “A microemulsion is defined as a system of water, oil, and amphiphile which is a single optically isotropic and thermodynamically stable liquid solution”. Just to emphasize the differences, “macroemulsions” or simply “emulsions” are not optically isotropic, are not thermodynamically stable, and are not spontaneously formed (require energy for the dispersion process). It should also be noted that a mixture of oil, water and amphiphile must be well-designed with respect of proper molecular structures and weight ratios to form a microemulsion (i.e., many oils and amphiphiles can form emulsions with water but only a few can form microemulsions).

Microemulsions have been studied as drug delivery systems on account of their solubilization capacity for poorly water-soluble drugs as well as potential for enhanced effect on topical and systemic drug bioavailability. Oral microemulsions have been successfully developed for cyclosporine to improve its oral bioavailability and by increasing it to reduce the absorption variations. U.S. Pat. No. 6,159,933, to Sherman, discloses an emulsion preconcentrate comprising a cyclosporine, dissolved in a solvent system of propylene carbonate and glycerides for mixing with gastrointestinal fluids, following oral administration.

In principle, non-oral extravascular administration of medications (such as transdermal and intranasal drug delivery) offers several advantages: elimination of variations in plasma concentration after gastrointestinal absorption, elimination of hepatic first pass metabolism, and avoidance of gastrointestinal intolerance. While topical drug delivery systems have been used for centuries for the treatment of local skin disorders, the use of the skin as a route for systemic drug delivery is of relatively recent origin.

Transdermal administration of drugs has been established in humans for nitroglycerine, estrogens, scopolamine, clonidine, testosterone, fentanyl and others. Transdermal or topical drug delivery systems based on microemulsions has been previously published (International patent applications WO 02/09763; WO 04/000358).

In WO 02/09763 there is described a transdermal delivery system for analgesic, anti-pyretic and anti-inflammatory drugs comprising an analgesic, anti-pyretic or anti-inflammatory drug in combination with water-miscible tetraglycol and water for dissolving said drug in hydrogel form.

In WO 04/000358, there is disclosed that not only non-steroidal-anti-inflammatory drugs can be effectively transported across the skin by the drug delivery system, but that many other types of active molecules may also be delivered transdermally utilizing a combination of water-miscible tetraglycol and water for dissolving such drugs in hydrogel form and this especially when said transdermal delivery system is in the form of a microemulsion prepared by mixing a drug model and tetraglycol. The obtained microemulsion resulted in an enhanced percutaneous permeation thus increased the drug's potential of curing, healing or improving its therapeutic effect.

Intranasal administration, similarly to transdermal administration, can avoid the inconveniences caused by injections into the body in connection with parenteral administration. However, unlike the transdermal route, intranasal administration may result in a rapid onset of effect, if required. Besides rapid absorption, the nasal route offers avoidance of hepatic first-pass metabolism, preferential drug delivery to brain via the olfactory region (Illum, J. Pharm. Pharmacol. 56: 3-17, 2004), and better compliance compared with injections done by untrained persons. Another advantage of using intranasal administration is the ability to deliver proteins and peptides into the systemic circulation, which otherwise could not be administered by routes other than parenteral injection.

Human insulin, for instance, has a very poor bioavailability via non-parenteral routes. Its bioavailability from a solution via the nasal route is also usually very poor. By using absorption enhancers (e.g., cyclopentadecalactone or CPE-215, Bentley Pharmaceuticals, Inc., North Hampton, N.H.), the systemic absorption still remains low, i.e., no more than 10-15%. Unlike large molecules, good results were obtained with small molecular weight polar drugs, which led to an increasing number of marketed products such as sumatripan (GlaxoSmithKline), zolmitripan (AstraZeneca), and butorphanol (Bristol Meyers Squibb).

Several other small molecules are under development, such as nasal morphine, nasal ketamine, for pain control, and nasal apomorphine for the treatment of erectile dysfunction (Illum, J. Control. Rel. 87: 187-198, 2003). Several medium molecular weight peptides have also been marketed, such as calcitonin (Novartis), buserelin (Aventis), and desmopressin (Ferring).

Unlike polar and water-soluble drugs, which can be delivered via nasal mucosa in aqueous solution, a large number of active substances are poorly or sparingly soluble in water and cannot be clinically applied as a nasal spray or nose-drops. It is also unwise to increase the volume of the nasal solution over approximately 200 microliters per nostril, due to immediate drainage of excess liquid toward the pharynx resulting in swallowing of most drug dosage.

Another example of the prior art is the intranasal administration of diazepam. This route for diazepam and benzodiazepines is a potential alternative to intravenous dosing in the treatment of acute epileptic seizures. One of the requirements for intranasal administration of diazepam is a very rapid onset of effect. Since the nasal delivery provides a means to circumvent the Blood-Brain Barrier and thus may allow increased CNS penetration of compounds, a more pronounced effect might be expected. Therefore, that entry into the systemic circulation of diazepam may not be the only indication for therapeutic drug effect (PK-PD relationship) following nasal instillation.

Over the last five years, only a few reports have been published describing vehicles for intranasal diazepam delivery (Bechgaard et al., J. Pharm. Pharmacol. 49: 747-750, 1997; Li et al., Int. J. Pharm. 199: 65-76, 2000, Int. J. Pharm. 237: 77-85, 2002; Lindhardt et al., 2001, 2002). PEG300 was used as a solubilizing vehicle for diazepam given to sheep, rabbit and man; however, its bioavailability was found to be relatively low (Lindhardt et al., Br. J. Clin. Pharmacol. 52: 521-527, 2001, Int. J. Pharm. 231: 67-72, 2002).

A second group (Li et al., Int. J. Pharm. 199: 65-76, 2000, Int. J. Pharm. 237: 77-85, 2002) has shown that alcohol-containing vehicles can significantly increase the bioavailability. Although it may be an effective absorption enhancer, alcohol causes irritation and soreness and its instillation into the nasal mucosa can lead to burning sensation, annoyance, and inconvenience.

International patent publication no. WO 91/16929 discloses a pharmaceutical composition wherein the drug (diazepam) is dissolved in a mixture of glycols for nasal administration.

Another international patent publication No. WO 90/02737 describes nasal administration of benzodiazepine hypnotics in a saline solution alcohol, glycol and glycol ether.

An earlier international patent publication no. WO 86/04233 discloses the drug (e.g., diazepam) in a mixture of propellant (e.g., halogenated hydrocarbon) and a solvent (e.g., glycerolphosphatide).

Despite the attention and interest in nasal drug delivery, only few publications have dealt with microemulsions as a formulative base for potential nasal products. Li et al. (Int. J. Pharm. 237: 77-85, 2002) described intranasal microemulsion formulation for diazepam prepared from Tween 80-propylene glycol-ethanol, water and oil (ethyl laurate). Zhang et al. (Int. J. Pharm. 275: 85-96, 2004) used several microemulsion combinations showing that a formulation containing Labrafil M/Cremophor RH40/ethanol combination was optimal for intranasal delivery of nimodipine comparing to formulations containing Labrasol/Transcutol combination.

Vyas et al. (J. Drug Target. 13: 317-324, 2005) described the use of Labrasol/Transcutol combination for zolmitripan, and in a later publication Vyas et al (J. Pharm. Sci. 95: 570-580, 2006) studied intranasal microemulsions of clonazepam based on a Cremophor/Tween 80/propylene glycol combination.

There is thus still a need to provide compositions for simple, non-irritant, and non-invasive external administration of known medicaments for human use which provide high bioavailability of the medicament.

### SUMMARY OF THE INVENTION

The present invention relates to pharmaceutical and cosmetic compositions for various purposes, which may be administered via a mucosal membrane or via transdermal, dermal and topical applications. The compositions are typically cosmetically or pharmaceutically acceptable and easy-to-apply systems containing drugs or other agents as active ingredients. More particularly, these systems composed of propylene carbonate in any system based on microemulsions or nano-sized emulsions.

In the present invention, it has now been found that various compositions or combinations, which are not based on glycols (such as macrogols, propylene glycol, tetraglycol, or Transcutol), may be successfully used as an effective drug delivery system.

Thus, according to the present invention there is now provided a transdermal, dermal, nasal and mucosal delivery system for a wide variety of drugs, as well as for polypeptides and protein-based drugs, in combination with oil, a wide variety of known surfactants, water-miscible propylene carbonate and water for dissolving said drugs in a microemulsion form.

The novel drug delivery system is preferably applied using an appropriate applicator and/or well-designed bio- and/or muco-adhesives, providing an effective and convenient mode of drug delivery to the skin, nostrils and mucous membranes.

In the practice of this invention a low-molecular, medium-molecular or high-molecular drug or a biologically active agent at concentrations from 0.0001% to 80% by weight are incorporated into pharmaceutically and/or cosmetically acceptable carriers such as liquids, cream, gel, spray, aerosol, foam, discs or patches. The resulting formulations can be re-applied several times daily to the skin surface, or onto the oral or the nasal mucosa of patients with various cosmetic or medical disorders, or any type of disease or pain. The medium contains any oil, surfactant, diluter and propylene carbonate at concentrations ranged for each from 0.001% to 99% by weight, and a gelling agent at concentrations ranged from 0% to 50% by weight.

The invention provides formulations that allow therapeutically efficient delivery of high concentrations of bioactive substances for absorption into cutaneous or mucosal tissues. The formulations according to the invention are generally non-irritating to the biological tissues, in spite of high concentrations, which may cause slight tingling of a passive nature.

According to another aspect of the invention, the formulations provide the further advantage of providing the biological active in a particle/droplet size that gets closer to the molecular size of the biological active. As the particle size decreases, the drug penetration/absorption increases.

The formulations according to the invention generally comprise one or more biologically active agents or combinations thereof, selected oils, water and selected surfactants in combination with propylene carbonate in a form that could be conveniently applied onto particular biological membranes. The term “biological membranes” means skin surface or the mucosa (mucous membrane) of the oral and nasal cavities.

There is thus provided according to some embodiments of the present invention, a transdermal, transmucosal pharmaceutical or cosmetic composition suitable for substantially extra-vascular application of at least one biologically active substance to biological membranes of a mammal, comprising:

a pharmaceutical or cosmetic composition comprising:
propylene carbonate;
at least one oil or source of fatty acid or surfactant; and
water; in combination with
the at least one biologically active substance;

wherein the propylene carbonate is adapted to enhance the bioavailability of the at least one biologically active substance.

According to some embodiments, the pharmaceutical or cosmetic composition is a pharmaceutical composition.

According to some embodiments, the pharmaceutical composition is a microemulsion or a nano-sized emulsion.

According to some further embodiments, the pharmaceutical composition comprises water-miscible propylene carbonate.

According to yet some further embodiments, the propylene carbonate is in a concentration of 0.001% to 99% weight/weight.

According to some embodiments, the at least one oil is selected from the group consisting of alkyl, dialkyl, trialkyl, acyl, diacyl, triacyl, monoglycerides, diglycerides and triglycerides of mono- di- or tri-carboxylic acids selected from the group consisting of saturated mono- di- or tri-carboxylic acids and mono- or di- or tri-carboxylic acids containing ethylenic unsaturation.

In some cases, the at least one oil or source of fatty acid is selected from amides, ethoxylated fats, mineral oil, petrolatum, vegetable oil, animal fats, and polyols.

According to some embodiments, the at least one oil or source of fatty acid is selected from isopropyl palmitate, isopropyl myristate, diethyl sebacate, diisopropyl adipate, cetyl oleate, oleyl alcohol, hexadecyl stearate, hexadecyl alcohol, caprylic triglycerides, capric triglycerides, isostearic triglycerides, adipic triglycerides, medium chain triglycerides (C8-C10 fatty acids), PEG-6-olive oil (Labrafil), propylene glycol myristyl acetate, lanolin oil, polybutene, wheatgerm oil, vegetable oils such as castor oil, corn oil, cottonseed oil, olive oil, palm oil, coconut oil, canola oil, sunflower oil, jojoba oil, peanut oil, hydrogenated vegetable oils, etc., and mineral oil.

In some examples, the pharmaceutical composition further comprises at least one muco-adhesive. According to some embodiments, the least one muco-adhesive is selected from acrylic polymers, polysaccharides, cellulose derivatives, cationized polymers, proteins, glycoproteins, and lectins.

According to some embodiments, the pharmaceutical composition further comprises at least one gelling agent. The at least one gelling agent may be selected from cationized guar gum, cellulose derivatives, acrylic polymers, polysaccharides, lipids, proteins, and polyhydroxy compounds. The at least one gelling agent may be present in a concentration of 0.01% to 50%.

According to some embodiments, the at least one surfactant is may be in a concentration of 0.1% to 90% wt./wt. The at least one surfactant may be selected from ionic or non-ionic surfactants. In some cases, the at least one surfactant is selected from bile salts and their derivatives thereof.

According to some embodiments, the at least one surfactant is selected from lecithin, lysolecithins, various phospholipids (e.g., phosphatidylcholine), oleic acid, and its derivatives thereof, fusidic acid and its derivatives thereof, polyoxyethylene alcohol ethers, polyoxyethylene sorbitan derivatives (polysorbates, e.g., Tweens such as Tween 20, 40, 60, 80, 85, etc.), sorbitan esters of fatty acids (e.g, sorbitan sesquioleate, sorbitan isostearate, sorbitan monolaurate, sorbitan monostearate, sorbitan monooleate, etc.), sugar esters (e.g., Sisterna sucrose esters, which are based on sucrose and vegetable fatty acids), capryloylcaproyl macrogol-8-glycerides (Labrasol), gelatine, albumin, starch, polyvinylpyrrolidone, polyvinyl alcohol, cetostearyl alcohol, glyceryl monoesters of fatty acids (e.g., glyceryl monostearate, glyceryl monooleate, glyceryl dioleate, etc.), polyglyceryl-6-dioleate (Plurol oleique), polyoxyethyleneglycol derivatives of fatty acids (e.g., Myrj 45, 49, 51, 52, 52S, 53, 59 etc.), polyoxyethyleneglycol ethers (e.g., polyoxyethylene (23) dodecyl ether or Brij 35 etc.).

According to some embodiments, the at least one biologically active substance is selected from an antibiotic, a polypeptide, a hormone, a protein-based drug, an anticancer an antiviral agent, a neurologically effective drug, an anti-emetic, an antihistamine, an anti-inflammatory agent, an anti-cholinergic drug an anti-hypertensive agent, an anti-angina drug, a narcotic analgesic, a narcotic antagonist, a blood factor, a bone metabolism agent, a prostaglandin, a protease inhibitor, an anti-parkinsonian drug, a combination of any of the biologically active substances or biologically active fragments or derivatives thereof.

In some notable cases, the at least one biologically active substance is insulin.

According to some embodiments, the pharmaceutical composition further comprises a pharmaceutically acceptable carrier.

According to some embodiments, the pharmaceutical composition may be in a form selected from a liquid solution, a cream, a lotion, a gel, a spray, an aerosol, foam, a disc and a dermal patch.

In some cases, the pharmaceutical micro-emulsion composition further comprises at least one of a stabilizer and a shape-forming agent. According to some embodiments, the at least one of a stabilizer and a shape-forming agent are selected from the group consisting of cationized guar gum, cellulose derivatives, acrylic polymers, polysaccharides, lipids, proteins, and polyhydroxy compounds.

The pharmaceutical composition, according to some embodiments may be suitable for application to a mucous membrane. In some cases, the mucous membrane may be located in the nasal cavity.

According to some embodiments, the composition is suitable for application by nasal spray or aerosol.

According to some embodiments, the pharmaceutical composition may be suitable for application by means of a nasal solution to be dripped into the nostrils.

According to some further embodiments, the pharmaceutical composition may be suitable for application by means of a nasal gel or ointment to be spread into the nostrils.

According to some embodiments, the mucous membrane may be located in the oral (or buccal) cavity.

According to some embodiments, the pharmaceutical composition may be suitable for application by oral spray or aerosol.

According to some further embodiments, the pharmaceutical composition may be suitable for application by means of a oral solution to be dripped or gargle in the mouth.

According to some embodiments, the pharmaceutical composition may be suitable for application by means of a oral gel or ointment to be spread onto the oral mucosa.

In some cases, the composition is suitable for topical application.

According to some embodiments, the pharmaceutical composition may be suitable for application by means of a dermal or transdermal patch.

There is thus provided according to some further embodiments of the present invention, a method for forming a transdermal, transmucosal pharmaceutical micro-emulsion composition suitable for substantially external application of at least one biologically active substance to biological membranes of a mammal, the method comprising:

admixing propylene carbonate, at least one oil or source of fatty acid, or at least one surfactant and water to form a micro-emulsion; and

adding the at least one biologically active substance to the micro-emulsion such that the propylene carbonate enhances the bioavailability of the at least one biologically active substance.

According to some embodiments, there is provided a pharmaceutical micro-emulsion composition, substantially as described herein.

There is thus provided according to yet some further embodiments of the present invention, a method for treating a disease or disorder in a mammalian subject comprising non-invasively administering the transdermal, transmucosal pharmaceutical micro-emulsion composition as described herein to the mammalian subject. According to some embodiments, the mammalian subject is human. According to some embodiments, the disease is diabetes.

According to some embodiments, the micro-emulsion composition may be administered via a route selected from transdermal, dermal, nasal, buccal and mucosal.

There is thus provided according to yet some further embodiments of the present invention, a method for treating a disease or disorder in a mammalian subject comprising administering a pharmaceutical micro-emulsion composition to the subject, substantially as described herein.

Furthermore, there is provided, according to yet some further embodiments of the present invention, use of a pharmaceutical micro-emulsion composition in the preparation of a medicament for treating a disease or disorder, substantially as described herein specification.

According to some embodiments, there is provided a transdermal, transmucosal pharmaceutical micro-emulsion composition for use as a medicament for treating a disease or a disorder.

Additionally, according to some further embodiments, there is provided a cosmetic or naturaceutical micro-emulsion composition for use as means to improve skin appearance, beauty, and health of the external parts of the human body, substantially as described herein.

### BRIEF DESCRIPTION OF THE DRAWINGS

The invention will now be described in connection with certain preferred embodiments with reference to the following illustrative figures so that it may be more fully understood.

In the drawings:

FIG. 1A shows the effects of intra-nasal administration of a) a micro-emulsion comprising 20 IU/ml insulin (ME20) (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin (pharmaco-kinetics) in a diabetic rabbit, according to some embodiments of the present invention;

FIG. 1B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose (pharmaco-dynamics) in a diabetic rabbit, according to some embodiments of the present invention;

FIG. 2A shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin in a second diabetic rabbit, according to some embodiments of the present invention;

FIG. 2B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose in a second diabetic rabbit, according to some embodiments of the present invention;

FIG. 3A shows the effects of intra-nasal administration of a) a micro-emulsion comprising 20 IU/ml insulin (ME20) (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin (pharmaco-kinetics) in a third diabetic rabbit, according to some embodiments of the present invention;

FIG. 3B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose (pharmaco-dynamics) in a third diabetic rabbit, according to some embodiments of the present invention;

FIG. 4A shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin in a fourth diabetic rabbit, according to some embodiments of the present invention;

FIG. 4B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose in a fourth diabetic rabbit, according to some embodiments of the present invention;

FIG. 5 shows the effects of intra-nasal administration of a) a microemulsion given at a dosage of 1 IU/kg insulin (filled grey circles and filled grey squares) and b) subcutaneous injection of a solution (1 IU/kg Lispro insulin) (filled black circles and filled black squares) upon individual plasma levels of insulin in two healthy rabbits, according to some embodiments of the present invention;

FIG. 6 shows the effects of intra-nasal administration of a) a microemulsion comprising 20 IU/ml insulin ME20 (filled diamonds) given at a dosage of 1 IU/kg b) a microemulsion comprising 50 IU/ml insulin ME50 given at a dosage of 1 IU/kg (filled triangles) and c) an aqueous solution given intranasally at a dosage of 1 IU/kg (filled squares) upon individual plasma levels of insulin in a diabetic rabbit, according to some embodiments of the present invention;

FIG. 7 shows the effects of the surfactants' ratio in intra-nasal administration of 1 mg/kg diazepam in the microemulsion system of the invention—Rabbit #1 received Formula D, while Rabbit #2 received Formula C—upon diazepam plasma levels, according to some embodiments of the present invention; and

FIG. 8 shows the effects of Lispro insulin pharmacokinetics after topical application of 2.2 IU/cm2 in a microemulsion (20 IU/ml), according to some embodiments of the present invention.

### DETAILED DESCRIPTION OF EXEMPLARY EMBODIMENTS

The present invention is directed to transdermal, transmucosal pharmaceutical micro-emulsion compositions suitable for substantially external application of at least one biologically active substance to biological membranes of a mammal, the composition comprising:
(i) a micro-emulsion comprising propylene carbonate, at least one oil or source of fatty acid, and water; in combination with
(ii) the at least one biologically active substance;
   such that the propylene carbonate is adapted to enhance the bioavailability of said at least one biologically active substance.

The biologically active substances may include a wide variety of drugs, as well as for polypeptides and protein-based drugs.

As stated hereinabove, the pharmaceutical micro-emulsion compositions of the present invention typically comprise an effective amount of:
(a) a water-miscible propylene carbonate, in a concentration range of 0.001%-99% wt./wt.);
(b) water in a concentration range of 0.5%-90% wt./wt.;
(c) at least one oil or source of fatty acid or at least one surfactant, in a concentration range of 0.1-90% wt./wt.

The at least one oil or source of fatty acid preferably comprises esters selected from the group consisting of alkyl, dialkyl, trialkyl, acyl, diacyl, triacyl, monoglycerides, diglycerides and triglycerides of mono- di- or tri-carboxylic acids selected from the group consisting of saturated mono- di- or tri-carboxylic acids and mono- or di- or tri-carboxylic acids containing ethylenic unsaturation. Besides esters, the oil phase may be selected from amides, ethoxylated fats, mineral oil, petrolatum, vegetable oil, animal fats, and polyols. The oil preferably suitable for use include, without limitation, isopropyl palmitate, isopropyl myristate, diethyl sebacate, diisopropyl adipate, cetyl oleate, oleyl alcohol, hexadecyl stearate, hexadecyl alcohol, caprylic triglycerides, capric triglycerides, isostearic triglycerides, adipic triglycerides, medium chain triglycerides (C8-C10 fatty acids), PEG-6-olive oil (Labrafil), propylene glycol myristyl acetate, lanolin oil, polybutene, wheatgerm oil, vegetable oils such as castor oil, corn oil, cottonseed oil, olive oil, palm oil, coconut oil, canola oil, sunflower oil, jojoba oil, peanut oil, hydrogenated vegetable oils, etc., and mineral oil.

The at least one surfactant may include one or more cosmetically- or pharmaceutically acceptable emulsifiers and/or surfactants and/or absorption promoters. The surfactants may be ionic as well as non-ionic including bile salts and their derivatives thereof, lecithin, lysolecithins, various phospholipids (e.g., phosphatidylcholine), oleic acid, and its derivatives thereof, fusidic acid and its derivatives thereof, polyoxyethylene alcohol ethers, polyoxyethylene sorbitan derivatives (polysorbates, e.g., Tweens such as Tween 20, 40, 60, 80, 85, etc.), sorbitan esters of fatty acids (e.g, sorbitan sesquioleate, sorbitan isostearate, sorbitan monolaurate, sorbitan monostearate, sorbitan monooleate, etc.), sugar esters (e.g., Sisterna sucrose esters, which are based on sucrose and vegetable fatty acids), capryloylcaproyl macrogol-8-glycerides (Labrasol), gelatine, albumin, starch, polyvinylpyrrolidone, polyvinyl alcohol, cetostearyl alcohol, glyceryl monoesters of fatty acids (e.g., glyceryl monostearate, glyceryl monooleate, glyceryl dioleate, etc.), polyglyceryl-6-dioleate (Plurol oleique), polyoxyethyleneglycol derivatives of fatty acids (e.g., Myrj 45, 49, 51, 52, 52S, 53, 59 etc.), polyoxyethyleneglycol ethers (e.g., polyoxyethylene (23) dodecyl ether or Brij 35 etc.).

In certain embodiments, at least one of the surfactants preferably comprises at least one esterified carboxylic group in its structure.

In addition, the compositions of the present invention preferably include further components as follows:

(e) gelling agents in a concentration range of 0.01%-50% wt./wt.)

Gelling agents may be incorporate in case where the microemulsion of the invention is required to be solidified in part in a form that the system could be applied conveniently on the biological membrane in a such way that it would not be removed or poured away. Preferable is the case when the gelling agent will enable adherence of the microemulsion sustem onto the application surface.

In the case where the composition according to the invention is a gel, soft or hard adhesive patch, stabilizers or shape-forming agents are selected from the group consisting of polymers such as cationized guar gum, cellulose derivatives, acrylic polymers, polysaccharides, lipids, proteins, and polyhydroxy compounds. The average molecular weight of these polymers can vary from 5,000 to 500,000 daltons;

(f) poly- or oligo-hyroxy compounds or their derivatives as co-solvents. These, compounds can be selected from the group of polyalkylene glycols, polyglyceryl of fatty acids (e.g., Plurol oleique), poloxamers, and di- or tri-ethylene glycol ethyl ethers, and sorbitol;

(g) preservatives such as parabens, phenoxyethanol, benzyl alcohol, and benzoic acid. Antioxidants selected from, without limitation, carnosine, carotenoids, lipoic acid, uric acid, urocanic acid, citric acid, lactic acid, glutathione, cysteine, thioredoxin, sulfoxamine compounds, selenium, ethylenediaminetetraacetic acid (EDTA) and its salts, ethylene glycol tetraacetic acid (EGTA), butylhydroxytoluene (BHT), butylhydroxyanisole (BHA), ubiquinone, ubiquinol and other quinines, vitamin C, ascorbyl derivatives, vitamin E, tocopherols and tocopherol derivatives, retinoids, vitamin A and its derivatives thereof, pH adjusting agents such as triethanolamine, citric and lactic acid may also be included in the composition.

The at least one biologically active substance may be selected from the group consisting of from an antibiotic, a polypeptide, a hormone, a protein-based drug, an anticancer an antiviral agent, a neurologically effective drug, an anti-emetic, an antihistamine, an anti-inflammatory agent, an anti-cholinergic drug an anti-hypertensive agent, an anti-angina drug, a narcotic analgesic, a narcotic antagonist, a blood factor, a bone metabolism agent, a prostaglandin, a protease inhibitor, an anti-parkinsonian drug, a combination of any of said biologically active substances or biologically active fragments or derivatives thereof.

Polypeptides or protein-based drugs or hormones may be selected from, but are not limited to, insulin, glucagons, follicle-stimulating hormone, growth hormone, vasopressin, adenocorticotropic hormone [ACTH], oxytocin, thyrotropin releasing hormone [TRH], luteinizing hormone releasing hormone [LHRH agonists such as leuprolide], and other analogs).

Aanticancer and antiviral agents may be selected from, but are not limited to, interferons (e.g., alpha2a,b-interferon, beta-interferon), anti-neoplastic agents (e.g., carmustine, doxorubicin, fluorouracil, cisplatin, cyclophosphamide, busulfan, carboplatin, leuprolide, megestrol, lomustine, levamisole, flutamide, etoposide, cytaranine, mitomycin, nitrogen mustard, paclitaxel, actinomycin, tamoxifen, vinblastine, vincristine, thiotepa, and chlorambucil, etc.).

Sex hormones may be selected from, but are not limited to, progesterone, estradiol-17-beta, testosterone, norethindrone, levonorgestrel, ethinylestradiol, FSH, luteinizing hormone [LH], etc.

Corticosteroids may be selected from, but are not limited to, hydrocortisone, prednisolone and budesonide.

Local anesthetics may be selected from, but are not limited to, lidocaine, prilocaine, benzocaine and tetracaine.

Neurologically effective drugs may be selected from, but are not limited to, anti-epileptics/anti-spasmolytics (e.g., benzodiazepines such as diazepam, clonazepam, lorazepam, etc.), and sedatives/tranquilizers (e.g., mirtazapine, trazodone, amobarbital, pentobarbital, secobarbital, alprazolam, clonazepam, diazepam, flunitrazepam, lorazepam, triazolam, chlorpromazine, fluphenazine, haloperidol, loxapine, perphenazine, prochlorperazine, thiothixene, trifluoperazine, clozapine, olanzapine, quetiapine, risperidone, ziprasidone, valerian, kava-kava, chloral hydrate, diethyl ether, eszopiclone, glutethimide, meprobamate, zolpidem, ramelteon, methyprylon, etc.), anti-depressants (e.g., imipramine, amoxapine, butriptyline, fluoxetine, sertraline, venlafaxine, citalopram, paroxetine, fluvoxamine, escitalopram, duloxetine, bupropion, amitriptyline, dosulepin, isocarboxazid, nialamide, pheneizine, selegiline, toloxatone, tranylcypromine, harmaline, iproclozide, iproniazid, clomipramine, desipramine, dibenzepin, dothiepin, Doxepin, iprindole, lofepramine, melitracen, nortriptyline, opipramol, protriptyline and trimipramine.

Anti-emetics may be selected from, but are not limited to, dopamine antagonists—metoclopramide, clopromazine, promethazine, domperidone, etc., serotonin antagonists—granisetron and ondansetron.

Antihistamines may be selected from, but are not limited to, cyclizine, promethazine, meclizine, and hydroxyzine.

Canabinoids may be selected from, but are not limited to, marinol and, cannabis. Further drugs include trimethobezamide and emetrol, amino acids and amino sugars (e.g., glucosamine, etc.).

Antibiotics may be selected from, but are not limited to, gentamycin, penicillin derivatives, streptomycin, aminoglycosides, cephalosporine, erythromycin and tetracycline.

Anti-inflammatory agents may be selected from, but are not limited to, steroidal—e.g., hydrocortisone, prednisone, prednisolone, triamcinolone, dexamethasone, betamethasone, beclomrthasone, clobetasone, clobetasol, budesonide, amcinonide, cortisone, desonide, flucinonide, flucinolone, methylprednisolone, mometasone, tixocortol, diflucortolone, diflorasone, halometasone, halcinonide, flucortolone, desoximetasone, etc., and nonsteroidal—e.g., acetylsalicylic acid, sasalate, ibuprofen, ketoprofen, naproxen, fenoprofen, flurbiprofen, oxaprozin, diclofenac, indomethacin, sulindac, tolmetin, piroxicam, meloxicam, mefenamic acid, nabumetone, etodalac, ketorolac, celecoxib, valdecoxib, and rofecoxib.

Anorectics may be selected from, but are not limited to, benzphetamine, diethylproprion, tepanilfenfluramine, mazindol, phendimetrazine, and phentermine.

Anti-allergic drugs may be selected from, but are not limited to, (e.g., antihistamines such as diphenhydramine, histamine, cromoglycate, meclizine and dimethindene maleate.

Anti-cholinergic drugs may be selected from, but are not limited to, scopolamine and atropine.

Parasympathomimetics may be selected from, but are not limited to, carbachol, bethanechol, nicotine, methacholine, pilocarpine, donepezil, edrophonium, physostigmine, pyridostigmine, neostigmine, tacrine, echothiophate, isofluorophate, cisapride, metoclopramide and sildenafil.

Antihypertensive agents may be selected from, but are not limited to, prazosin, propranolol, timolol, metoprolol, pindolol, labetalol, guanethidine, reserpine, methyldopa, guanabenez, clonidine, nifedipine, captopril, enalapril, lisinopril, verapamil, diltiazem, thiazides, furosemide, hydralazine, minoxidil and nitroprusside.

Anti-angina drugs may be selected from, but are not limited to, nicardipine, nadolol, diltiazem, isosorbide mononitrate, isosorbide dinitrate, metoprolol, nitroglycerine, amlodipine, nifedipine and atenolol.

Narcotic analgesics may be selected from, but are not limited to, morphine, codeine, heroin and methadone.

Narcotic antagonists may be selected from, but are not limited to, naloxone and naltrexone.

Anti-asthma/bronchodilatorsors may be selected from, but are not limited to, albuterol/salbutamol, ephedrine, metaproterenol, terbutaline, epinephrine, theophylline, ipratropium, salmeterol, fluticasone, formoterol, beclomethasone and fluticasone.

Blood factors may be selected from, but are not limited to, factor VII, VIII, and IX.

Bone metabolism agents may be selected from, but are not limited to, calcitriol (vitamin D3) and alendronate.

Prostaglandins may be selected from, but are not limited to, alprostadil, dinoprost, latanoprost and misoprostol.

Protease inhibitors include aprotinine.

Antiparkinsonian agents may be selected from, but are not limited to, levodopa, carbidopa, amantadine, selegiline, entacapone, biperiden, benserazide and apomorphine.

Various dyes and diagnostic agents, and combinations of such agents are also within the scope of the present invention.

In certain embodiments of the invention, the biologically active substance is a saccharide, amino acid, nucleotides (ribonucleotides and deoxyribonucleotides), small peptide, including without limitation, carnosine, N-acetyl-cysteine, N-acetyl-D-glucosamine, N-acetyl-carnithine, methionine, ascorbates (vitamin C and its derivatives), vitamin E and its derivatives thereof, vitamin B12, vitamin B6, folic acid, carotenoids (e.g., beta carotene, lycopene, astaxanthine, cantaxanthine etc.), niacin, taurine or combinations thereof.

In other embodiments, the biologically active substance is a biological additive, a term indicating any compound obtained from a natural source, including plants, animals, bacteria, fungi, and yeast, which has a medicinal or any beneficial effect when applied to human body. These may include extracts of Chamomile, Aloe Vera, Ashwaghanda, Papaya, Propolis, rose Hip, Walnut, Witchhazel, (Hamamelis), Fenugreek, Ginseng, Gingko, etc. Other biological or biotechnological agents may be medicinal microorganisms or cellular biomasses, such as Cordyceps spp., □ Ganoderma spp., □ and Monascus spp. (Red yeast).

Preferred transdermal compositions according to the present invention are cosmetically- or pharmaceutically accepted and easy-to-apply skin-adhesive systems containing active ingredient/s. More particularly, these systems composed of propylene carbonate as a co-surfactant, which assist in dissolving or solubilizing the active materials in a microemulsion containing oil, water and surfactant/s, and facilitating their penetration through the lipophilic strata of the skin.

Preferred intranasal compositions according to the present invention are cosmetically- or pharmaceutically accepted and easy-to-apply muco-adhesive (i.e., containing adhesive polymers such as carbopol and polycarbophil) or regular systems containing active ingredient/s. More particularly, these systems composed of propylene carbonate as a co-surfactant, which assist in dissolving or solubilizing the active materials in a microemulsion containing oil, water and non-ionic surfactant/s, and facilitating their penetration through the lipophilic strata of the nasal mucous membranes.

While the invention will now be described in connection with certain preferred embodiments in the following examples and with reference to the accompanying figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims.

Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

The following examples demonstrate the invention:

### EXAMPLE 1

### Preparation of Micro-Emulsions

Micro-emulsions, having compositions as exemplified, but not limited to the examples in the tables hereinbelow, were prepared for example by the following methods:

### 1. Liquid Microemulsion

a) A required amount of a water-soluble drug is dissolved in 20 g water containing 0.1% benzyl alcohol (preservative). In a separate vessel 10 g isopropyl palmitate (or myristate), 14.6 g glyceryl oleate, 11.67 g propylene carbonate, and Labrasol are mixed well. Then the aqueous solution is added and mixed by a magnetic stirrer or an electrical mixer (e.g., Heidolph mixer). The micro-emulsion is stored at 4° C. or room temperature for further use
b) 20 g water containing 0.1% benzyl alcohol or benzoic acid is mixed with 10 g isopropyl palmitate (or myristate), 14.6 g glyceryl oleate, 11.67 g propylene carbonate, and Labrasol. Then, the drug is added and mixed by a magnetic stirrer or an electrical mixer (e.g., Heidolph mixer) until completely dissolved. The micro-emulsion is stored at 4° C. or room temperature for further use
c) A required amount of a drug is dissolved in 11.67 g propylene carbonate. While mixing, 10 g isopropyl palmitate (or myristate), 14.6 g glyceryl oleate, and Labrasol are added and mixed until a clear solution is obtained. Then, 20 g water containing 0.1% benzyl alcohol (preservative) are added and mixed by a magnetic stirrer or an electrical mixer (e.g., Heidolph mixer) until one phase liquid ios formed. The obtained micro-emulsion is stored at 4° C. or room temperature for further use

### EXAMPLE 2

### Solid Microemulsion Preparation for the Purpose of Dermal or Transdermal Patch

a) In a 200-ml vessel, 10 g of isopropyl palmitate (or myristate), 14.05 g glyceryl oleate, 11.25 g propylene carbonate, 42.2 g Sisterna PS750, and 19.5 or 15 g water were mixed together using a high speed stirrer such as a Heidolph mixer at a low speed for 5 minutes. A drug (e.g. 3 or 7.5 g lidocaine base) was added and dissolved in the microemulsion for 15 minutes at the same speed. After complete dissolution, 50 g of Jaguar C162 were added and mixed for 30 more minutes at a low speed. The gelled micro-emulsion was stored in special circle-shaped molds at room temperature to form a patch.
b) In a 200-ml vessel, 10 g of isopropyl palmitate (or myristate), 7.8 g glyceryl oleate, 6.25 g propylene carbonate, 23.45 g Labrasol, and 49.5 or 45 g water were mixed together using a high speed stirrer such as a Heidolph mixer at a low speed for 5 minutes. A drug (e.g. 3 or 7.5 g lidocaine base) was added and dissolved in the microemulsion for 15 minutes at the same speed. After complete dissolution, 50 g of Jaguar C162 were added and mixed for 30 more minutes at a low speed. The gelled micro-emulsion was stored in special circle-shaped molds at room temperature for overnight to form a patch.

### EXAMPLE 3

### Microemulsion Semi-Solid Preparation for Dermal or Nasal Gel

In a 200-ml vessel, 10 g of isopropyl palmitate (or myristate), 37.5 g glyceryl oleate, 25 g propylene carbonate, 12.5 g Labrasol, and 9 g water were mixed together using a high speed stirrer such as a Heidolph mixer at a low speed for 5 minutes. A drug (e.g. 5 g diazepam) was added and dissolved in the microemulsion for 30 minutes at the same speed. After complete dissolution, 1 g of carbopol 934 was added and mixed for 30 more minutes at a low speed. The micro-emulsion gel was stored in jars or tubes for further use.

### COMPARATIVE EXAMPLE 1

### Intranasal Delivery of Insulin

This example is a study performed in vivo using a rabbit model as described below:

The study was designed to evaluate the pharmacokinetics (PK) as well as the activity of short-acting human insulin after nasal administration, using a novel microemulsion preparation, containing 20 IU/ml (ME20) and 50 IU/ml (ME50) concentrations of the hormone and nontoxic pharmaceutically-acceptable inactive ingredients as described (in % w/w) in the table (Table 1) hereinbelow:

A cross-over study was performed in healthy and diabetic white rabbits, which were provided subcutaneously (SC) (1 or 0.5 IU/kg, respectively) and intra-nasal (IN) doses (1 IU/kg) with a wash-out time of at least two days between the administrations.

An ELISA assay was run to determine drug plasma levels during a 4-hr period of pharmacokinetic monitoring. Plasma glucose levels were measured at predetermined time intervals to follow up the hypoglycemic effect.

TABLE 1
Composition of two different micro-emulsions, ME-20 and ME- 50.

| | ME 20 | ME 50 |
|---|---|---|
| | (% wt/wt) | (% wt/wt) |
| Isopropyl palmitate (oil) | 10 | 10 |
| Glyceryl oleate (Surfactant) | 14.59 | 8.33 |
| Propylene carbonate (Co-S) | 11.67* | 6.67* |
| Labrasol (Surfactant) | 43.74 | 25 |
| Lispro solution for injection | 20 | 50 |
| (Humalog R 100 IU/ml, Eli Lilly)** | | |

*Co-surfactant/surfactants (Co-S/S) = 0.2
**Humalog solution for injection containing a preservative, m-cresol. Also note that the insulin solution is used the aqueous phase. Normally, insulin and the preservative are first dissolved in water at a required concentration, and the obtained solution is added into the oil/surfactant mixture.

The tested preparations were:
Intranasal insulin microemulsion: the preparation contained 20 IU/ml or 50 IU/ml of Lispro insulin (Humalog R. Eli Lilly, IN). The microemulsion formulations were prepared without insulin and kept at room temperature. Prior to each experiment, insulin solution was added and mixed gently until a clear liquid was obtained
Insulin solution for injection: contained 100 IU/ml Lispro Insulin, (Humalog R 100), manufactured by Eli Lilly, Indianapolis, Ind. Prior to subcutaneous injection the solution for injection was diluted with saline.

Pharmacokinetic study: All animal procedures were performed in accordance with protocols approved by the Institutional Ethical Committee. Five New Zealand white rabbits (Hsdlf: NZW males, 2 kg, about 2-2.5 months old, Harlan, Jerusalem) were studied in a cross-over design with a wash-out period of at least two days. They were housed individually with free access to food and water. A 12 h light/12 h dark cycle was held to keep a normal circadian rhythm in the animals.

Before each experiment, food was deprived from the animals for 14-16 hours. Venflon™ cannula (22G, Becton Dickinson, Sweden) was inserted into the main artery of the rabbit ear, and blood was sampled for glucose determination until baseline levels were obtained. Each rabbit was weighed and was administered with 0.5 or 1 IU/kg SC dose (in approx. 0.5 ml solution) and 1 IU/kg IN dose (approx. 40 ?I of ME50 and 100 ?I of ME20). When 40 ?I of microemulsion was administered, the liquid was applied with a micropipette or sprayed into one nostril. When 100 ?I of microemulsion was administered, 50 ?I liquid was applied with a micropipette or sprayed into each nostril. The exact application volume was determined according to the individual body weight. Spraying technique was developed by using a 100 ?I syringe connected to MAD Nasal Drug Delivery Device (Wolfe Tory Medical, Inc., Salt Lake City, Utah) Blood samples were generally collected at 0, 2, 5, 15, 30, 45, 60, 90, 120, 180 and 240 minutes after application in heparin-treated tubes. Plasma was obtained after centrifugation at 10,000 rpm for 10 minutes, and stored at −20° C. until analyzed.

Alloxan-induced diabetes in rabbits: Rabbits were left for 24 hours without food. The fasted rabbits were anesthetized by IM injection of a combination of 15 mg/kg ketamine and 9 mg/kg xylazine. Diabetes was induced by iv injection of 60 mg/kg alloxan (in sodium citrate buffer, pH 4.0). The animals were kept overnight with food and water containing 5% glucose. Diabetes was determined after at least 2 days if animals had fasted blood glucose levels of above 300 mg/dL.

Plasma analysis for insulin and glucose: Plasma glucose levels were measured by glucose oxidase (GOD) method (Roche/Hitachi GOD-PAP test kit). Insulin was determined by enzyme-linked immunosorbent assay (Iso-Iinsulin ELISA, DRG International, Inc., USA).

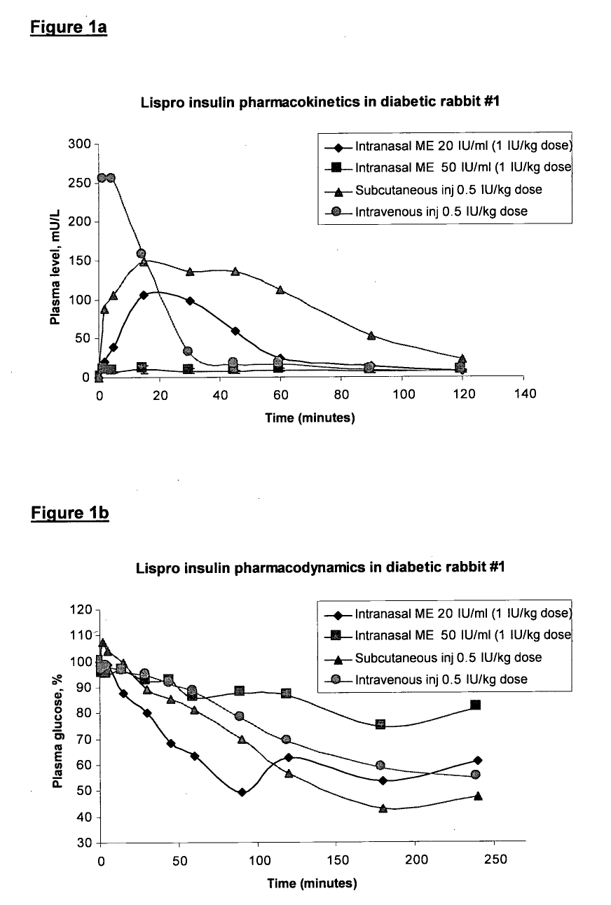

FIG. 1A shows the effects of intra-nasal administration of a) a micro-emulsion comprising 20 IU/ml insulin (ME20) (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin (pharmacokinetics) in a diabetic rabbit, according to some embodiments of the present invention;

FIG. 1B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose (pharmacodynamics) in a diabetic rabbit, according to some embodiments of the present invention;

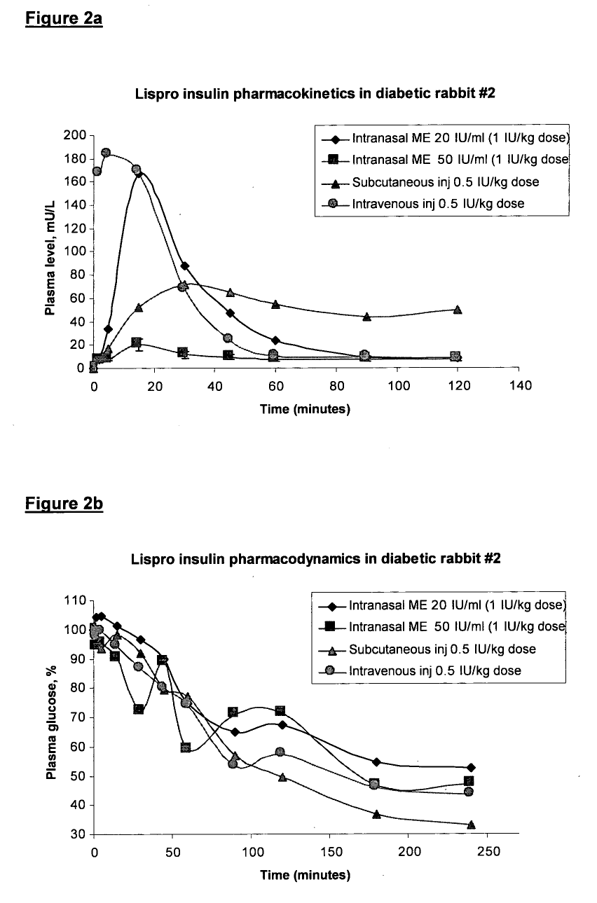

FIG. 2A shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin in a second diabetic rabbit, according to some embodiments of the present invention;

FIG. 2B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose in a second diabetic rabbit, according to some embodiments of the present invention;

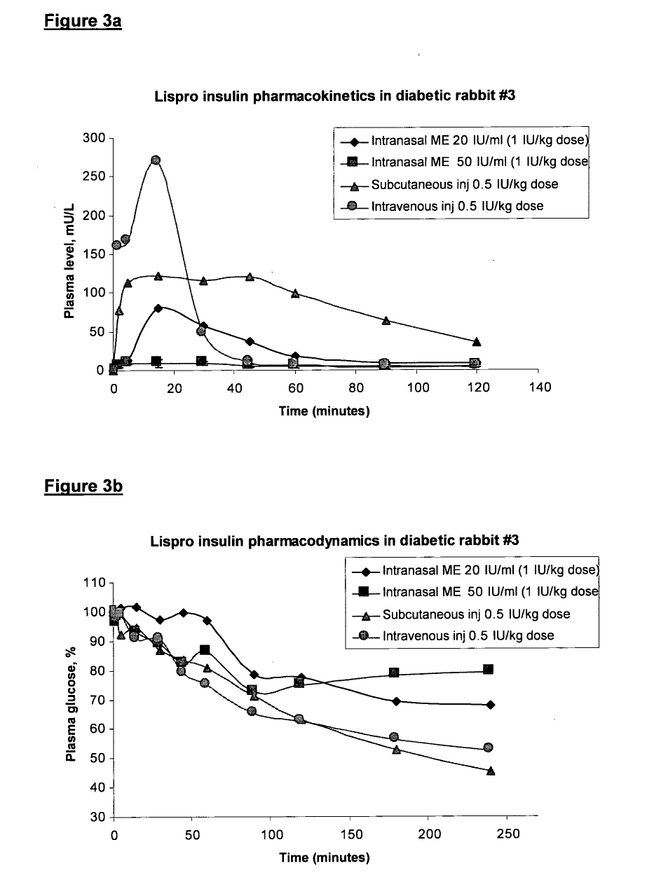

FIG. 3A shows the effects of intra-nasal administration of a) a micro-emulsion comprising 20 IU/ml insulin (ME20) (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin (pharmaco-kinetics) in a third diabetic rabbit, according to some embodiments of the present invention;

FIG. 3B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose (pharmaco-dynamics) in a third diabetic rabbit, according to some embodiments of the present invention;

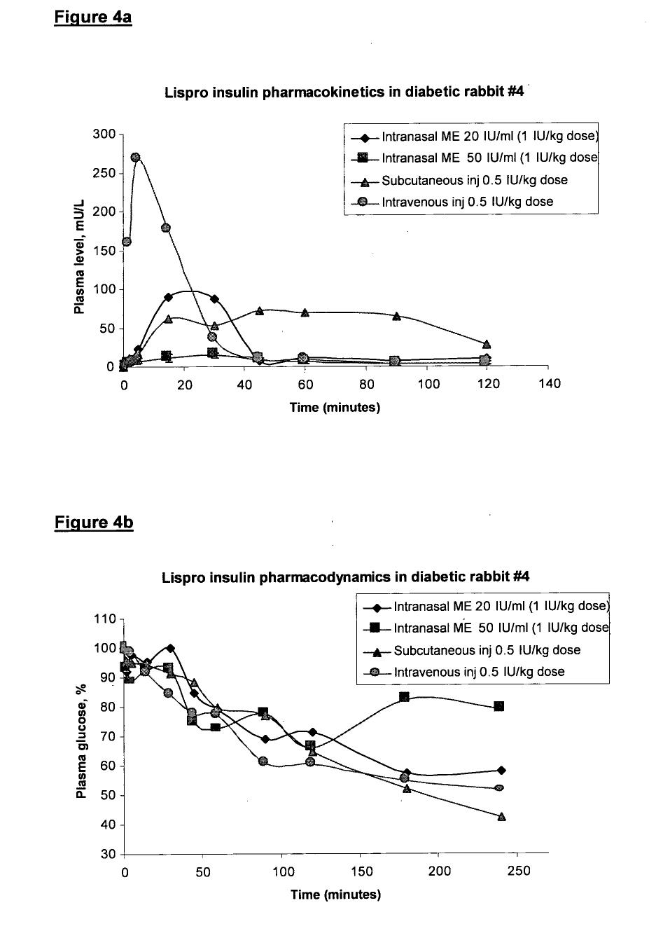

FIG. 4A shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of insulin in a fourth diabetic rabbit, according to some embodiments of the present invention;

FIG. 4B shows the effects of intra-nasal administration of a) a microemulsion comprising 1 U/kg insulin ME20 (filled diamonds) and a microemulsion comprising 50 IU/ml insulin (ME50) (filled squares) given at a dosage of 1 IU/kg as compared to: b) subcutaneous injection of a solution (0.5 IU/kg insulin) (filled triangles) and c) intravenous injection of a solution (0.5 IU/kg insulin) (filled circles) upon individual plasma levels of glucose in a fourth diabetic rabbit, according to some embodiments of the present invention;

At a disease stage: FIGS. 1A, 1B, 2A, 2B, 3A, 3B, 4A and 4B present the individual plasma levels (of insulin and glucose) obtained after SC and IN administration of insulin to diabetic rabbits. The figures also include the concomitant hypoglycemic response to these different routes of administration.

Table 2 summarizes the pharmacokinetic parameters obtained for the four diabetic animals by using the WinNonlin program (Professional version 4.1, Pharsight Corporation, Mountain View, Calif.). It can be seen that peak insulin plasma levels of 106.5 ?IU/ml 167.3 ?IU/ml, 79.5 ?IU/ml, and 89.8 ?IU/ml in rabbit #1, #2, #3 and #4, respectively, were reached after 15 minutes from the time of nasal application. Interestingly, these values were comparable to (in rabbit #1 and #4), lower than (in rabbit #3) and higher than (in rabbit #2) the respective values obtained after SC administration. However, the percentages of bioavailability obtained from these diseased rabbits by IN administration of ME20 were 41.2%, 70.6%, 27.5% and 33.0% (mean value=43.1%), which are, to the best of our knowledge, significantly higher values than any value published in the literature thus far.

The pharmacodynamic response of this route of administration, i.e., approximately 50% reduction in plasma levels similar to SC administration, reflects the similar peak plasma levels rather than the bioavailability obtained by the nasal route.

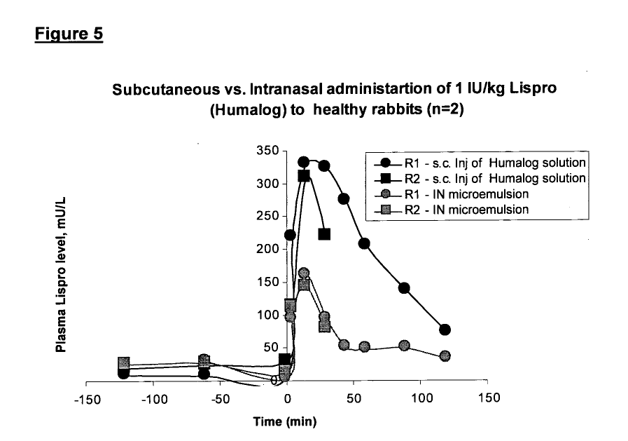

FIG. 5 shows the effects of intra-nasal administration of a) a microemulsion given at a dosage of 1 IU/kg insulin (filled grey circles and filled grey squares) and b) subcutaneous injection of a solution (1 IU/kg Lispro insulin) (filled black circles and filled black squares) upon individual plasma levels of insulin in two healthy rabbits, according to some embodiments of the present invention.

At a healthy stage: In a different studies performed before the diabetic animal model was taken place, IN application (spraying) of the micro-emulsion in healthy rabbits reveals somewhat higher bioavailability values than at the disease stage (see FIG. 5 and Table 3). Without being bound to any theory, this finding may be explained by that at a stage of diabetes there is less circulation of the mucosal blood vascularization at the site of application.

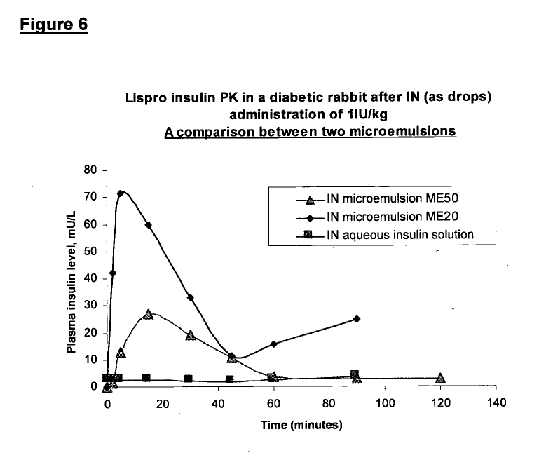

FIG. 6 shows the effects of intra-nasal administration of a) a microemulsion comprising 20 IU/ml insulin ME20 (filled diamonds) given at a dosage of 1 IU/kg b) a microemulsion comprising 50 IU/ml insulin ME50 given at a dosage of 1 IU/kg (filled triangles) and c) an aqueous solution given intranasally at a dosage of 1 IU/kg (filled squares) upon individual plasma levels of insulin in a diabetic rabbit, according to some embodiments of the present invention.

Influence of formulation: As shown in FIG. 6 and FIGS. 1-4, any changes in the microemulsion composition may influence the pharmacokinetics of insulin absorption through the nasal cavity. In the same diabetic rabbit, the nasal application of ME20, which contained 20% Humalog R solution for injection, was compared with the same mode of application of ME50, which contained 50% Humalog solution for injection. The peak plasma level and the relative bioavailability increased significantly when the microemulsion was prepared with 20% insulin solution. That means that more effective administration can be achieved by decreasing the concentration of insulin in the formula and increasing the volume of the liquid dose.

### Definition of Pharmacokinetic Parameters as Appearing in the Tables:

PK parameter

Cₘₐₓ (mIU/L) = peak plasma drug concentration
tₘₐₓ (min) = time to reach peak plasma drug concentration
?_{z} (lambda_{z}) (min − 1) = the linear terminal slope obtained by plotting the logarithmic
values of plasma drug levels versus time.
Elimination t_{1/2} (min) = the total body half-life elimination of drug
AUC_{0-∞} (mIU · min · L⁻¹) = Area-under-the-curve of plasma drug level versus time plot
from time zero to infinity.
AUC_{0-∞}/dose (mIU · min · L⁻¹ · D⁻¹) = Area-under-the-curve of plasma drug level versus
time plot from time zero to infinity, normalized by the dose.
F_{R}^{a} (%) = Relative bioavailability, i.e., Fraction absorbed in relation to another
extravascular administration.
F_{A}^{b} (%) = Absolute bioavailability, i.e., Fraction absorbed in relation to intravenous
administration (IV data).

TABLE 2
Pharmacokinetic parameters of insulin after IV, SC and IN administration to diabetic
rabbits

| | IV* | SC | IN | IV* | SC | IN |
|---|---|---|---|---|---|---|
| PK parameter | 0.5 IU/kg | 0.5 IU/kg | 1 IU/kg | 0.5 IU/kg | 0.5 IU/kg | 1 IU/kg |
| | Diabetic Rabbit #1 | | | Diabetic Rabbit #2 | | |
| Cₘₐₓ (mIU/L) | 253.7 | 148.4 | 106.5 | 183.1 | 71.6 | 167.3 |
| tₘₐₓ (min) | 2 | 15 | 15 | 5 | 30 | 15 |
| ?_{z} (min−1) | .0113 | 0.0263 | 0.0189 | 0.03 | 0.0047 | 0.0026 |
| Elimination t_{1/2} | 61.1 | 26.3 | 36.7 | 23.1 | 149.0 | 271.1 |
| (min) | | | | | | |
| AUC_{0-∞} | 6638.6 | 12192.7 | 5473.1 | 6145.1 | 16772.0 | 8675.1 |
| (mIU · min · L⁻¹) | | | | | | |
| AUC_{0-∞}/dose | 13277.2 | 24385.5 | 5473.1 | 12290.2 | 33544.1 | 8675.1 |
| (mIU · min · L⁻¹ · D⁻¹) | | | | | | |
| F_{R}^{a} (%) | 100 | 100 | 22.4 | 100 | 100 | 25.9 |
| F_{A}^{b} (%) | 100 | 100 | 41.2 | 100 | 100 | 70.6 |
| | Diabetic Rabbit #3 | | | Diabetic Rabbit #4 | | |
| Cₘₐₓ (mIU/L) | 268.3 | 121.5 | 79.5 | 267.2 | 72.5 | 89.8 |
| tₘₐₓ (min) | 15 | 15 | 15 | 5 | 45 | 15 |
| ?_{z} (min−1) | 0.0097 | 0.0164 | 0.0235 | 0.0148 | 0.0119 | 0.0218 |
| Elimination t_{1/2} | 71.7 | 42.3 | 29.4 | 46.8 | 58.4 | 31.9 |
| (min) | | | | | | |
| AUC_{0-∞} | 6628.2 | 12798.8 | 3644.5 | 5816.2 | 9130.1 | 3845.4 |
| (mIU · min · L⁻¹) | | | | | | |
| AUC_{0-∞}/dose | 13256.4 | 25597.7 | 3644.5 | 11632.4 | 18260.2 | 3845.4 |
| (mIU · min · L⁻¹ · D⁻¹) | | | | | | |
| F_{R}^{a} (%) | 100 | 100 | 14.2 | 100 | 100 | 21.0 |
| F_{A}^{b} (%) | 100 | 100 | 27.5 | 100 | 100 | 33.0 |

^{a}F_{R} % = relative bioavailability = (AUC_{0-∞}^{IN} × Dose^{SC}) × 100/(AUC_{0-∞}^{SC} × Dose^{IN})
^{a}F_{A} % = absolute bioavailability = (AUC_{0-∞}^{IN} × Dose^{IV}) × 100/(AUC_{0-∞}^{IV} × Dose^{IN})
*To avoid insulin shock, the dose was gradually injected intravenously for 4-5 minutes (not an IV bolus)
Mean absolute bioavailability (F_{A}) = 43.1% (±19.2%)

TABLE 3
Pharmacokinetic parameters of insulin after
SC and IN administration to healthy rabbits

| | Healthy Rabbit #1 | | Healthy Rabbit #2 | |
|---|---|---|---|---|
| PK parameter | SC 1 IU/kg | IN 1 IU/kg | SC 1 IU/kg | IN 1 IU/kg |
| Cₘₐₓ (mIU/L) | 329.5 | 160.4 | 309.2 | 141.6 |
| tₘₐₓ (min) | 15 | 15 | 15 | 15 |
| ?z (min − 1) | 0.0165 | 0.0128 | — | — |
| Elimination t_{1/2} (min) | 42.0 | 53.9 | — | — |
| AUC_{0-∞} (mIU · min · L⁻¹) | 28952.3 | 10391.5 | 6426.8^{b} | 3206.9^{b} |
| AUC_{0-∞}/dose | 28952.3 | 10391.5 | 6426.8^{b} | 3206.9^{b} |
| (mIU · min · L⁻¹ · D⁻¹) | | | | |
| F^{a} (%) | 100 | 35.9 | 100 | 49.9 |

^{a}F % = relative bioavailability = (AUC_{0-∞}^{IN} × Dose^{SC}) × 100/(AUC_{0-∞}^{SC} × Dose^{IN})
^{b}AUC₀₋ₗₐₛₜ = AUC₀₋₃₀

### Mean Relative Bioavailability (F)=42.9%

### COMPARATIVE EXAMPLE 2

### Intranasal Delivery of Diazepam

This example is a study performed in vivo using a rabbit model as described below:

The study was designed to evaluate the bioavailability of diazepam (an anticonvulsive and skeletal muscle relaxant) after intranasal administration, using a microemulsion preparation of the present invention. The nasal preparation (INDM=intranasal diazepam microemulsion) contained 5% (w/w) concentration of the active principal and nontoxic pharmaceutically-acceptable inactive ingredients as described below. A cross-over study was performed in three white rabbits, which were administered intravenously (IV) and IN doses (1 mg/kg) with a wash-out time of at least 7 days between the administrations. A sensitive HPLC assay was run to determine drug plasma levels during a 4-hr period of pharmacokinetic monitoring.

The tested preparations were:
intranasal diazepam microemulsions (INDM): The preparations contained 5% or 50 mg/g of diazepam USP (vendor batch no. 0309010003, F.I.S Fabrica, Italy; Expiration date: June 2008). The microemulsion formulations were kept at room temperature.
Assival solution for injection: Contained 10 mg/2 ml (0.5%) diazepam, Lot # 057233 (Expiration Date: June 2007), manufactured by Teva Pharmaceutical Industries, Israel.

Pharmacokinetic study: Two New Zealand white rabbits (Hsdlf: NZW males, 2.5 kg, about 3-month old, Harlan, Jerusalem) were studied for diazepam bioavailability in a cross-over design with a wash-out period of at least 7 days. They were housed individually with free access to food and water. Just before the experiment, a Venflon™ cannula (22G, Becton Dickinson, Sweden) was inserted into the main artery of the rabbit ear, and blood was collected for time zero. Each rabbit was weighed and was administered with 1 mg/kg dose IV (approx. 0.5 ml of Assival 5 mg/ml solution) and 1 mg/kg IN (approx. 0.05 ml of 5% INDM).

TABLE 4
Compositions of four different micro-emulsions (A-D)

| | Formula | Formula | Formula | Formula |
|---|---|---|---|---|
| | A | B | C | D |
| Isopropyl | 10 | 10 | 10 | 10 |
| palmitate | | | | |
| Glyceryl | 33.3 | 16.7 | 37.5 | 25 |
| oleate | | | | |
| Propylene | 25* | 25* | 25* | 25* |
| carbonate | | | | |
| Labrasol | 16.7 | 33.3 | 12.5 | 25 |
| Diazepam | 5 | 5 | 5 | 5 |
| Water | 10 | 10 | 10 | 10 |
| Surfactants' | 1:2 | 2:1 | 1:3 | 1:1 |
| ratio** | | | | |

*Co-surfactant/surfactants = 0.5
**Labrasol/glyceryl oleate ratio

In the IV treatment, the rabbits received diazepam through the marginal vein of the ear that was not used for collecting blood. The bolus administration lasted over 20 s. In the IN treatment, each rabbit received about 25 microliters of INDM into each nostril with a micropipette. The exact application volume was determined according to the individual body weight. Blood samples (2 ml) were collected at 2, 5, 10, 20, 30, 45, 60, 120, 180, and 240 minutes after application in heparin-treated tubes. Plasma was obtained after centrifugation at 10,000 rpm for 10 minutes, and stored at −20° C. until analyzed.

HPLC analysis of diazepam in plasma: The plasma samples were analyzed by HPLC and UV detection. Prior to injection the plasma (250 □I) was mixed with 250 ?I 0.01% (v/v) perchloric acid solution in acetonitrile. After centrifugation at 10,000 rpm for 10 minutes, the supernatant solution was transferred into 1.5 ml amber vials.

Aliquots of 100 ?I from each vial were injected into HPLC system [Shimadzu VP series including LC-10AT pump, a SCL-10A system controller, an auto-injector (SIL-10AD), degasser (DGU-14A), and SPD-M10A diode-array detector for peak spectrum identification], equipped with a prepacked C₁₈ column (Betasil C18, 5?m, 250×4.6 mm, ThermoHypersil, UK) heated to a temperature of 40° C. The quantitation of diazepam was performed by integration of peaks detected at 230 nm. The samples were chromatographed using an isocratic mobile phase consisting of phosphate buffer (pH 3.5)-methanol-acetonitrile (40:50:10) at a flow rate of 1 ml/min. A calibration curve (peak area versus drug concentration) was constructed by running calibration curve for every series of chromatographed samples. The calibration series composed of rabbit plasma spiked with standard diazepam (batch No 0309010003, F.I.S Fabrica Italy), which underwent the same treatment as the unknown plasma samples. Calibration curves were linear over the range of 0.2-2 ?g/ml (0.2, 0.4, 0.8, 1, 1.5, and 2?g/ml in plasma).

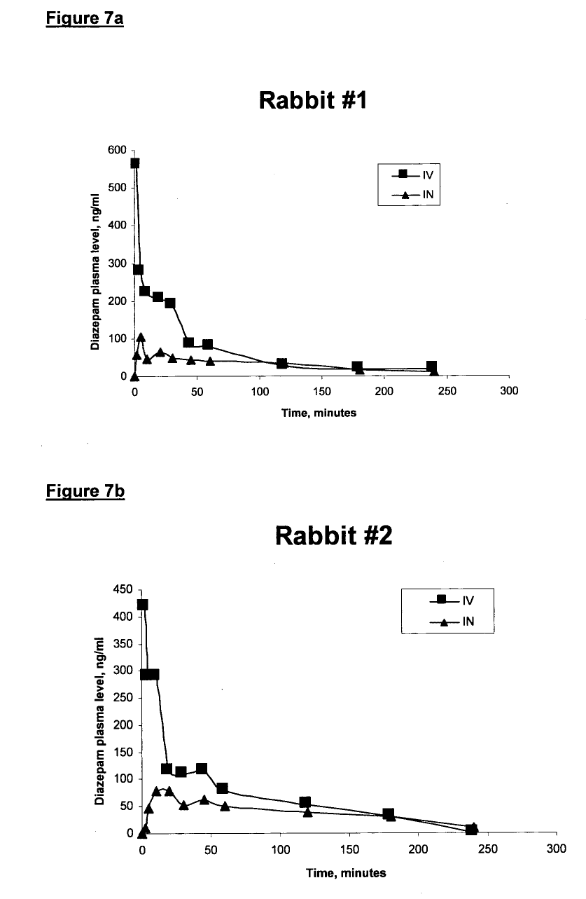

FIG. 7 shows the effects of the surfactants' ratio in intra-nasal administration of 1 mg/kg diazepam in the microemulsion system of the invention—Rabbit #1 received Formula D, while Rabbit #2 received Formula C) upon diazepam plasma levels, according to some embodiments of the present invention.

### COMPARATIVE EXAMPLE 3

### Topical Lidocaine Formulations

| | MEL 20a | MEL 20b | MEL 50 |
|---|---|---|---|
| | (% wt/wt) | (% wt/wt) | (% wt/wt) |
| Isopropyl Palmitate | 10 | 10 | 10 |
| Glyceryl oleate | 14.05 | 15.34 | 7.8 |
| Propylene carbonate | 11.25* | 6.14** | 6.25* |
| Labrasol | — | — | 23.45 |
| Sisterna PS750 | 42.2 | 46.02 | — |
| Water | 20 | 20 | 50 |
| Lidocaine base | 2.5 | 2.5 | 2.5 |

*Co-surfactant/surfactants = 0.2
**Co-surfactant/surfactants = 0.1

Skin Lidocaine Depot (in ?g/cm²) Obtained after Application of 100 Microliter/cm² Microemulsion onto Rats In Vivo (4≦n≦10)

| Test Formulation | Drug concentration in the applied skin |
|---|---|
| MEL 20a | 24.37 ± 6.83 ?g/cm² |
| MEL 20b | 24.02 ± 13.00 ?g/cm² |
| MEL 50 | 25.41 ± 4.35 ?g/cm² |
| Control (plain solution) | 2.19 ± 0.98 ?g/cm² |

Three formulations of microemulsion containing 2.5% lidocaine was formulated as described in the above table. Aliquots of 200 microliter of each formulation was applied in vivo to rats after anesthesia (1.8 cm2 application surface area, abdominal skin). After 1 hour, the remaining drug was removed from the skin surface by swabbing, washing and tape-stripping (10 times), and drug was extracted and analyzed by HPLC assay. As shown in this example, no difference was found between the three microemulsions in delivering lidocaine into the skin layers, however, it was obvious that the microemulsion system was superior over a plain, non-particulate system. This demonstrates one aspect of the novelty of this invention.

### COMPARATIVE EXAMPLE 4

### Topical Insulin Formulations

In order to demonstrate the ability of microemulsion of this invention to deliver insulin through the skin into the plasma, the following microemulsion formulation was prepared and tested on rat abdominal skin in vivo:

| | ME 20 (% wt/wt) |
|---|---|
| Isopropyl palmitate | 10 |
| Glyceryl oleate | 14.59 |
| Propylene carbonate | 11.67* |
| Labrasol | 43.74 |
| Lispro solution for injection | 20 |
| (Humalog R 100 IU/ml, Eli Lilly) | |

*Co-surfactant/surfactants (Co-S/S) = 0.2

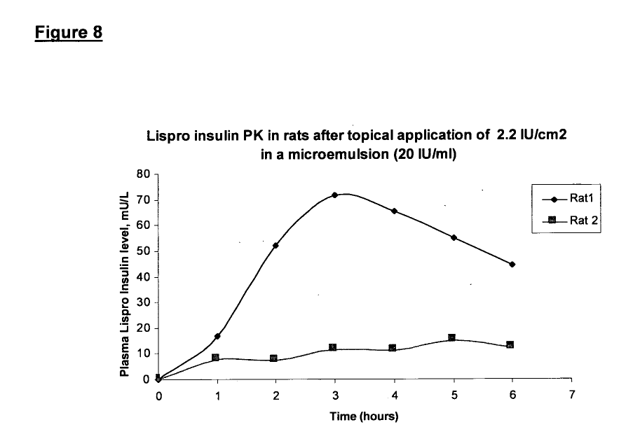

FIG. 8 shows the effects of Lispro insulin pharmacokinetics after topical application of 2.2 IU/cm² in a micro-emulsion (20 IU/ml), according to some embodiments of the present invention. As shown, at least in one animal of the two tested significantly elevated hormone levels were noted with a peak plasma level reached after three hours.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

It should be understood, that by the term “comprise” as used in the present invention is meant that various other inactive ingredients, compatible drugs and medicaments can be employed in the compositions as long as the critical propylene carbonate are present in the compositions and are used in the manner disclosed.

All percentages herein are by weight unless otherwise specified. It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A transdermal, transmucosal pharmaceutical composition suitable for substantially extra-vascular application of at least one biologically active substance to biological membranes of a mammal, comprising:
pharmaceutical or cosmetic composition comprising:
propylene carbonate;
at least one oil or source of fatty acid or surfactant; and
water; in combination with the at least one biologically active substance;
wherein the propylene carbonate is adapted to enhance the bioavailability of said at least one biologically active substance.

2. A pharmaceutical composition according to claim 1, wherein the composition is a microemulsion or a nano-sized emulsion.

3. A pharmaceutical composition according to claim 1, wherein the propylene carbonate is water-miscible.

4. A pharmaceutical composition according to claim 1, wherein the propylene carbonate is in a concentration of 0.001% to 99% weight/weight.

5. A pharmaceutical composition according to claim 1, wherein the at least one oil is selected from the group consisting of alkyl, dialkyl, trialkyl, acyl, diacyl, triacyl, monoglycerides, diglycerides and triglycerides of mono- di- or tri-carboxylic acids selected from the group consisting of saturated mono- di- or tri-carboxylic acids and mono- or di- or tri-carboxylic acids containing ethylenic unsaturation.

6. A pharmaceutical composition according to claim 1, wherein the at least one oil or source of fatty acid is selected from amides, ethoxylated fats, mineral oil, petrolatum, vegetable oil, animal fats, and polyols.

7. A pharmaceutical composition according to claim 1, wherein the at least one oil or source of fatty acid is selected from isopropyl palmitate, isopropyl myristate, diethyl sebacate, diisopropyl adipate, cetyl oleate, oleyl alcohol, hexadecyl stearate, hexadecyl alcohol, caprylic triglycerides, capric triglycerides, isostearic triglycerides, adipic triglycerides, medium chain triglycerides (C8-C10 fatty acids), PEG-6-olive oil (Labrafil), propylene glycol myristyl acetate, lanolin oil, polybutene, wheatgerm oil, vegetable oils such as castor oil, corn oil, cottonseed oil, olive oil, palm oil, coconut oil, canola oil, sunflower oil, jojoba oil, peanut oil, hydrogenated vegetable oils, etc., and mineral oil.

8. A pharmaceutical composition according to claim 1, further comprising at least one muco-adhesive.

9. A pharmaceutical composition according to claim 8, wherein the at least one muco-adhesive is selected from acrylic polymers, polysaccharides, cellulose derivatives, cationized polymers, proteins, glycoproteins, and lectins.

10. A pharmaceutical composition according to claim 1, further comprising at least one gelling agent.

11. A pharmaceutical micro-emulsion composition according to claim 10, wherein the at least one gelling agent is selected from cationized guar gum, cellulose derivatives, acrylic polymers, polysaccharides, lipids, proteins, and polyhydroxy compounds.

12. A pharmaceutical composition according to claim 1, wherein the at least one gelling agent is present in a concentration of 0.01% to 50%.

13. A pharmaceutical composition according to claim 1, wherein the at least one surfactant is in a concentration of 0.1% to 90% wt./wt.

14. A pharmaceutical composition according to claim 13, wherein the at least one surfactant is selected from ionic or non-ionic surfactants.

15. A pharmaceutical composition according to claim 13, wherein the at least one surfactant is selected from bile salts and their derivatives thereof.

16. A pharmaceutical composition according to claim 13, wherein the at least one surfactant is selected from lecithin, lysolecithins, various phospholipids (e.g., phosphatidylcholine), oleic acid, and its derivatives thereof, fusidic acid and its derivatives thereof, polyoxyethylene alcohol ethers, polyoxyethylene sorbitan derivatives (polysorbates, e.g., Tweens such as Tween 20, 40, 60, 80, 85, etc.), sorbitan esters of fatty acids (e.g, sorbitan sesquioleate, sorbitan isostearate, sorbitan monolaurate, sorbitan monostearate, sorbitan monooleate, etc.), sugar esters (e.g., Sisterna sucrose esters, which are based on sucrose and vegetable fatty acids), capryloylcaproyl macrogol-8-glycerides (Labrasol), gelatine, albumin, starch, polyvinylpyrrolidone, polyvinyl alcohol, cetostearyl alcohol, glyceryl monoesters of fatty acids (e.g., glyceryl monostearate, glyceryl monooleate, glyceryl dioleate, etc.), polyglyceryl-6-dioleate (Plurol oleique), polyoxyethyleneglycol derivatives of fatty acids (e.g., Myrj 45, 49, 51, 52, 52S, 53, 59 etc.), polyoxyethyleneglycol ethers (e.g., polyoxyethylene (23) dodecyl ether or Brij 35 etc.).

17. A pharmaceutical composition according to claim 1, wherein the at least one biologically active substance is selected from an antibiotic, a polypeptide, a hormone, a protein-based drug, an anticancer an antiviral agent, a neurologically effective drug, an anti-emetic, an antihistamine, an anti-inflammatory agent, an anti-cholinergic drug an anti-hypertensive agent, an anti-angina drug, a narcotic analgesic, a narcotic antagonist, a blood factor, a bone metabolism agent, a prostaglandin, a protease inhibitor, an anti-parkinsonian drug, a combination of any of said biologically active substances or biologically active fragments or derivatives thereof.

18. A pharmaceutical composition according to claim 17, wherein the at least one biologically active substance is insulin.

19. A pharmaceutical composition according to claim 1, further comprising a pharmaceutically acceptable carrier.

20-33. (canceled)

34. A method for forming a transdermal, transmucosal pharmaceutical micro-emulsion composition suitable for substantially external application of at least one biologically active substance to biological membranes of a mammal, the method comprising:
admixing propylene carbonate, at least one oil or source of fatty acid, or at least one surfactant and water to form a micro-emulsion; and
adding the at least one biologically active substance to the micro-emulsion such that the propylene carbonate enhances the bioavailability of said at least one biologically active substance.

35-43. (canceled)
